(19) United States
(12) Patent Application Publication
(10) Pub. No.: US 2014/0039540 A1
(43) Pub. Date: Feb. 6, 2014

(54) MEDICAL DEVICES INCLUDING BLOOD CLOT REMOVING MEDICAL DEVICES, AND METHODS OF USING SAME

(71) Applicant: Richard B. Park, St. Charles, IL (US)

(72) Inventor: Richard B. Park, St. Charles, IL (US)

(21) Appl. No.: 13/565,333

(22) Filed: Aug. 2, 2012

(51) Int. Cl. A61B 17/22; A61M 29/00

(52) U.S. Cl. 606/200; 606/191

(57) ABSTRACT

Medical devices and methods for removing blood clots or debris are disclosed. In an embodiment, a blood clot removing device includes a sheath having a size for inserting the sheath into a blood vessel and having a flexible end portion. An actuator is coupled to a dilator and configured to expand the flexible end portion of the sheath. A wire is moveable within an inner passageway of the sheath. An inflatable balloon is coupled to the wire and is configured such that when the balloon is inflated, a blood clot can be captured within the expanded flexible end portion of the sheath and removed from the blood vessel.

### BACKGROUND

The present disclosure relates generally to medical devices and methods of using medical devices, and more specifically to blood clot or debris (including medical devices such as a lost stent) removing medical devices and methods of using same.

Blood vessel clots, or thrombosis, are typically removed via an invasive surgical procedure involving a large incision. A balloon tipped wire is inserted into a blood vessel and the balloon is placed at a position beyond the location of the blood clot. The balloon is inflated and the wire and the inflated balloon are pulled backwards towards the large incision site to remove the clot or debris (e.g., a lost stent).

A need exists for medical devices and methods that improve the process of removing blood clots or debris, and that do not require invasive surgical incision procedures.

### SUMMARY

The present disclosure is directed generally to medical devices and methods for removing blood clots or debris percutaneously, without requiring a large and invasive surgical incision. In an embodiment, a medical device includes a sheath, a dilator, an actuator and a wire. The sheath is constructed for insertion into a blood vessel of a patient, and includes a flexible end portion. The dilator is moveable within a passageway defined in the sheath, and the actuator is configured such that when it is activated or actuated, the actuator causes the flexible end portion of the sheath to expand. A wire having an inflatable balloon is moveable within an inner passageway of the sheath. The inflatable balloon is configured so that it can be placed in a deflated state at a position beyond the location of the blood clot or debris. When the deflated balloon is placed beyond the location of the blood clot, the balloon is inflated and both the balloon and the wire are pulled generally backwards towards the site at which the sheath was inserted into the patient's blood vessel so that the balloon pulls and captures the blood clot within the expanded flexible end portion of the sheath. With the blood clot captured inside the expanded flexible end portion of the sheath, the blood clot is removed by pulling both the wire and the sheath out of the patient through the insertion site.

In an embodiment, the actuator is an inflatable balloon coupled to the dilator, and the inflation of the balloon causes the flexible end portion of the sheath to expand. In an embodiment a divider separates the dilator into a first inner passageway and a second inner passageway. One of the first and second inner passageways is configured to allow air or pneumatic pressure to inflate the balloon coupled to the dilator, and the other of the first and second passageways is configured to allow the wire to be moveable and insertable within the dilator.

In another embodiment, the actuator causes the flexible end portion of the sheath to expand so that at least a portion of the expanded flexible end portion conforms generally to the shape of the blood vessel. The expanded flexible end portion of the sheath contacts an inner wall of the blood vessel. In one such embodiment, at least a portion of the expanded flexible end portion is generally cylindrical, conical and/or frusto-conical in shape. The flexible end portion in an embodiment has a thickness that allows the flexible end portion to expand due to a force of caused by the inflation and expansion of the balloon actuator. In an embodiment, the thickness of the flexible end portion is less than the remaining or adjacent portion of the sheath to allow the flexible end portion to expand and remain in a substantially cylindrical, conical and/or frusto-conical like shape after it has expanded. In an alternative embodiment, the flexible end portion includes a plurality of pedals that expand or splay outwardly to retain the expanded shape of the flexible end portion.

In another embodiment, a blood clot removing device includes a sheath having a flexible end portion. The sheath is sized for insertion into a blood vessel, and defines an inner passageway that allows a dilator to be moved within the inner passageway. The flexible end portion of the sheath is expandable by an actuator so that a blood clot can be captured within the expanded flexible end portion, and removed from the blood vessel. In an embodiment, the inner passageway is a first inner passageway, and the sheath further defines a second inner passageway that allows a dilator to be moved within the second inner passageway. The flexible end portion is located at an end section of one of the first and second inner passageways, and the sheath can be used in combination with, or independent of, removing a blood clot, such as for inserting instruments into one or both of the first and second inner passageways for operating or performing a procedure on a patient.

In another embodiment a blood clot removing method includes inserting a sheath into a blood vessel, inserting a wire into the sheath, inflating a balloon coupled to the wire, expanding a flexible end portion of the sheath, pulling the wire along with the inflated balloon so as to capture a blood clot in the expanded flexible end portion of the sheath, and pulling both the sheath and the wire to remove the captured blood clot.

It is accordingly an advantage of the present disclosure to provide medical devices and methods that simplify and improve procedures for removing blood clots or thrombosis.

It is a further advantage of the present disclosure to reduce the need for invasive surgical procedures in treating or removing blood clots and debris.

It is additionally an advantage of the present disclosure to provide medical devices and procedures that reduce the cost and time associated with treating and removing blood clots or debris.

It is yet another advantage of the present disclosure to provide medical devices and methods that provide a single sheath having at least two passageways or lumens and that operates similar to two separate sheaths, but does not require a large insertion site like two separate sheaths.

Additional features and advantages are described herein, and will be apparent from the following Detailed Description and the Figures.

### BRIEF DESCRIPTION OF THE FIGURES

FIG. 1A illustrates an embodiment of the present disclosure including a dilator and an actuator.

FIG. 1B illustrates an embodiment of the present disclosure including a sheath having a flexible end portion.

FIG. 1C illustrates a cross-section of the sheath of FIG. 1B taken along line A-A.

FIG. 1D illustrates an embodiment of the present disclosure including a sheath, a dilator and a wire.

FIG. 1E illustrates an embodiment of the present disclosure including a sheath, a dilator and a wire, wherein a flexible end portion of the sheath is in an expanded or activated state.

FIGS. 2A to 2D illustrate an embodiment of a blood clot or debris removing method using a medical device of the present disclosure.

FIGS. 3A to 3D illustrate another embodiment of the present disclosure including a sheath defining first and second inner passageways.

### DETAILED DESCRIPTION

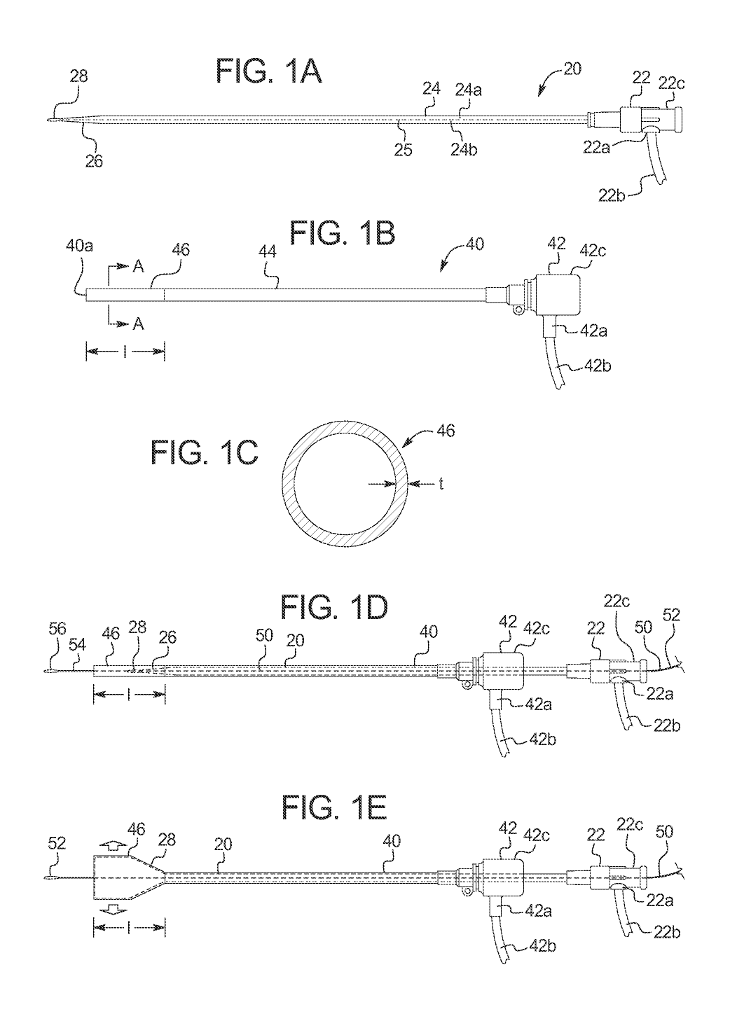

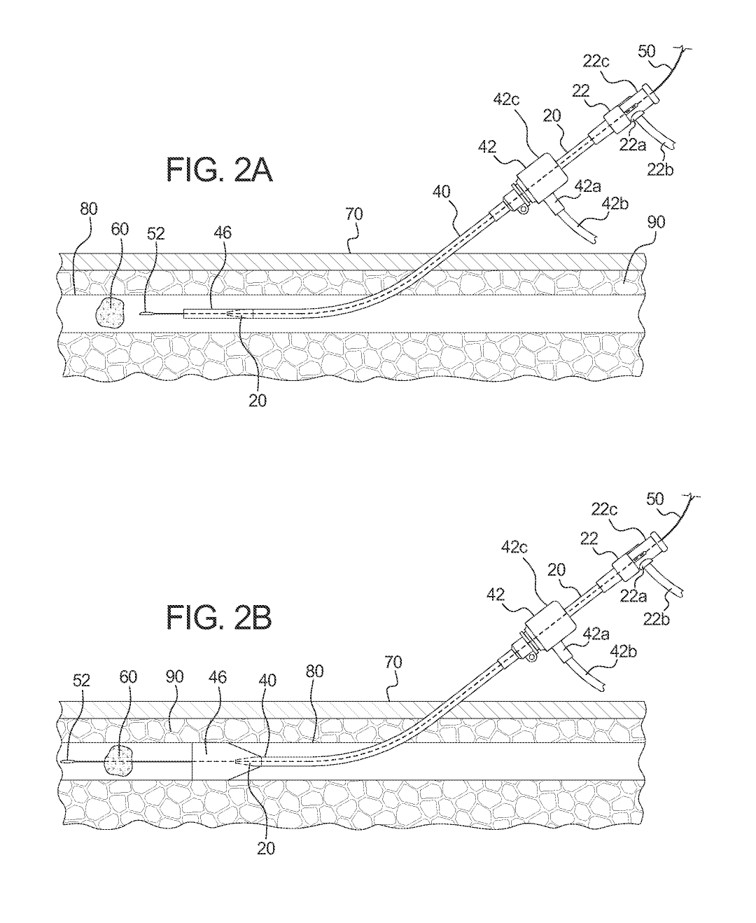

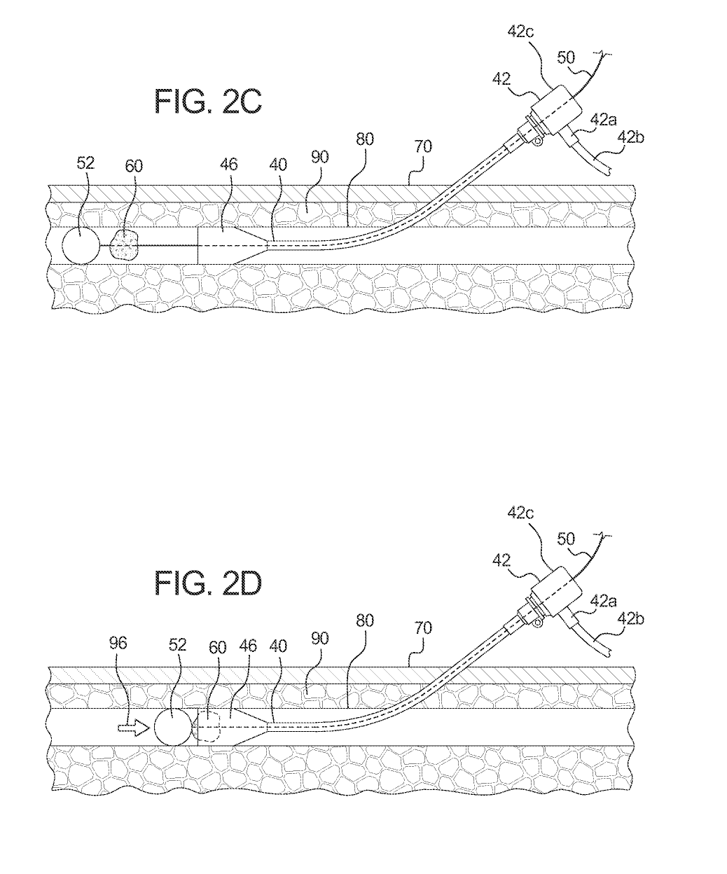

Turning now to the drawings, FIGS. 1A to 1D illustrate an embodiment of a blood clot or debris removing medical device of the present disclosure. FIGS. 2A to 2D illustrate an embodiment of a method of removing a blood clot or debris using a medical device of the present disclosure.

Referring more specifically to FIGS. 1A to 1E, an embodiment of a medical device of the present disclosure having three primary components is illustrated, namely, a dilator 20, a sheath 40, and a wire 50. FIG. 1A illustrates dilator 20 including a body 24 having a proximal portion 22 and a distal or end portion 26. Body 24 includes a divider 25 separating dilator 20 into first 24a and second 24b inner passageways extending along the length of dilator 20. Proximal portion 22 includes a port 22a and a tube 22b coupled to a valve apparatus 22c. Tube 22b and port 22b are configured so as to allow air or pneumatic fluid pressure to be delivered and received through tube 22b and port 22b, into one of the first 24a and second 24b inner passageways. An actuator 28 in the form of an inflatable balloon is coupled to proximal or end portion 26 of dilator 20. It should be appreciated that actuator 28 can be coupled to any portion of dilator, and that in different embodiments, actuator 28 is coupled to an instrument different from dilator 20, such as to a wire described below.

FIG. 1B illustrates sheath 40 defining an internal passageway 40a. Sheath 40 includes a body 44 having a proximal portion 42 and a distal or flexible end portion 46. Proximal portion 42 includes a port 42a and a tube 42b and a valve apparatus 42c. Internal passageway 40a of sheath 40 has an inner diameter that is less than or equal to the outer diameter of dilator 20, which allows dilator 20 to be insertable and moveable within the internal passageway 40a defined in sheath 40. FIG. 1C is a cross-section of distal flexible portion 46 taken along line A-A of FIG. 1B, and shows a thickness t of flexible end portion 46. Thicknesses t of flexible end portion 46 enables the entire flexible end portion 46 to flex and expand due to the expansive force of an inflatable balloon actuator as described below. It should be appreciated that the thickness t of flexible portion 46 can be any suitable thicknesses that allows flexible portion 46 to expand and retain an expanded shape, so as to be able to capture a blood clot in the expanded flexible end portion (as discussed below). For example, the thickness t of flexible end portion 46 can range from about 0.01 mm to 0.5 mm. It should also be appreciated that the thickness t of end portion 46 in an embodiment is less than the thickness of the adjacent portion and/or the remaining portion of sheath 40. For example, the thickness of remaining or adjacent portion of sheath 40 in an embodiment can range from about 0.5 mm to 15 mm. Flexible end portion 46 also includes a length 1. In an embodiment, the length 1 ranges from about 10 mm to 30 mm, however, it should be appreciated that flexible end portion 46 can be any suitable length sufficient to capture a blood clot within the expanded flexible end portion 46 as described below.

In an embodiment, thickness t of flexible end portion 46 varies along the length of flexible end portion 46. For example, flexible end portion 46 in an embodiment includes a proximal section having a smaller thickness than the remaining or distal section of flexible end portion 46. With a same outward force applied to both sections, the smaller thickness of the proximal section allows the proximal section to flex and expand outwardly a greater distance than the distal section expands or flexes, so as to form a conical or frusto-conical like shape or portion for capturing and retaining a blood clot in the flexible expanded end portion, as illustrated and described below.

Referring to FIG. 1E, dilator 20 is illustrated at a position in which it has been inserted into the inner passageway of sheath 40, and distal portion 26 has been located at least partially within flexible end portion 46 of sheath 40. With dilator 20 inserted into sheath 40 and distal portion 26 located at least partially within flexible end portion 46 of sheath 40, inflatable balloon actuator 28 is actuated to expand flexible end portion 46 such that an outer diameter of flexible end portion 46 increases to approximately the same size as the diameter of an inner wall of a blood vessel as illustrated and described below in connection with FIGS. 2A to 2D.

FIG. 1E illustrates wire 50 having been inserted into dilator 20 and sheath 40. Wire 50 includes a proximal portion 52 and a distal or end portion 54. An inflatable balloon 56 is coupled to the distal or end portion 54 and inflated. It should be appreciated that wire 50 can be any suitable size so as to be insertable and moveable within one (or both) of first 24a and second 24b passageways defined in dilator 20.

Turning now to FIGS. 2A to 2D, an embodiment of a method for removing or treating a blood clot or thrombosis using a medical device of the present disclosure is illustrated. In particular, to remove a blood clot or thrombosis, a blood vessel is located by feeling around on a patient's skin. When the blood vessel is located, a needle is inserted into the patient's skin and into the blood vessel. A wire (not illustrated and separate from the wire described in connection with FIGS. 1A to 1D above) is then inserted into the needle and into the blood vessel. The needle is removed from the patient, and a sheath and dilator are placed over the wire and into the blood vessel using the rail as a guide. The wire and the dilator are then removed from the patient, and a new wire having an inflatable balloon coupled to the wire is inserted into the sheath.

FIG. 2A illustrates the point in time in which the sheath, the dilator, and the new wire (including the inflatable balloon coupled to the wire) have been inserted into the blood vessel. In particular, FIG. 2A illustrates an embodiment of a medical device of the present disclosure in which the medical device has been inserted into (i) the patient's skin 70, (ii) the patient's tissue 90 located below the skin 70, and (ii) the patient's blood vessel 80, so that a clot 60 located within the blood vessel 80 can be removed. The medical device in this embodiment includes dilator 20, sheath 40 and wire 50. Dilator 20 has been inserted into the passageway 40a defined in sheath 40, and extends along the length of sheath 40. Balloon actuator 28 (not shown at FIG. 2A) has been located internal to sheath 40, and is in a deactivated or deflated state. Wire 50, including balloon 52 coupled thereto, has been inserted into one of the passageways 24a, 24b defined in dilator 20.

When the medical device has been inserted into the patient's blood vessel 80 and the inflatable balloon 52 of wire 50 has been moved to a position beyond the location of the clot or debris 60, the actuator balloon 28 of dilator 20 is inflated. FIG. 2B illustrates the actuation and inflation of balloon 52 causing flexible end portion 46 of sheath 40 to expand outwardly. Balloon 28 is actuated by flowing air or gas through dilator tube 22b, into one of dilator passageways 24a, 24b to inflate balloon actuator 28. The inflation of balloon actuator 28 expands balloon 28 outwardly, which exerts an outward force onto an inner surface of flexible end portion 46. The outward force flexes and expands end portion 46 radially outwardly, thereby increasing the diameter of flexible end portion 46 so that at least a portion of end portion 46 contacts the inner wall of the blood vessel. At least a portion of the flexible end portion 46 accordingly expands to form a shape similar to the shape of the patient's blood vessel, thereby defining a cylindrical, conical and/or frusto-conical like shape.

FIG. 2C illustrates balloon 52 having been placed beyond the location of clot 60, flexible end portion 42 having been expanded via the activation or actuation of dilator balloon 28, and dilator 50 having been removed from sheath 40. FIG. 2D illustrates wire 50 being pulled backwardly (i.e., in the direction of arrow 96 at FIG. 2D), causing balloon 52 located at the tip of wire 50 to pull and capture blood clot or debris 60 in the internal, expanded end portion 46 of sheath 40. With blood clot 60 captured at least substantially within the expanded flexible end portion 46 of sheath 40, both sheath 40 and wire 50 can be pulled together out of the patient's skin to remove blood clot or debris 60 from the patient.

It should be appreciated from the foregoing that a method of removing a blood clot from a blood vessel includes inserting a sheath into a blood vessel, inserting a wire into the sheath, inflating a balloon coupled to the wire, expanding a flexible end portion of the sheath, pulling the wire along with the inflated balloon so as to capture a blood clot in the expanded flexible end portion of the sheath, and pulling both the sheath and the wire to remove the captured blood clot.

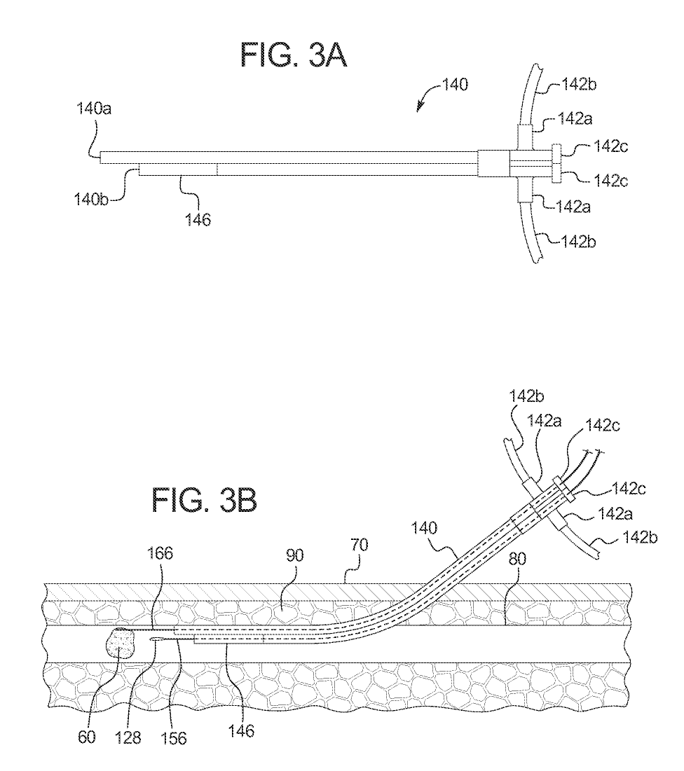

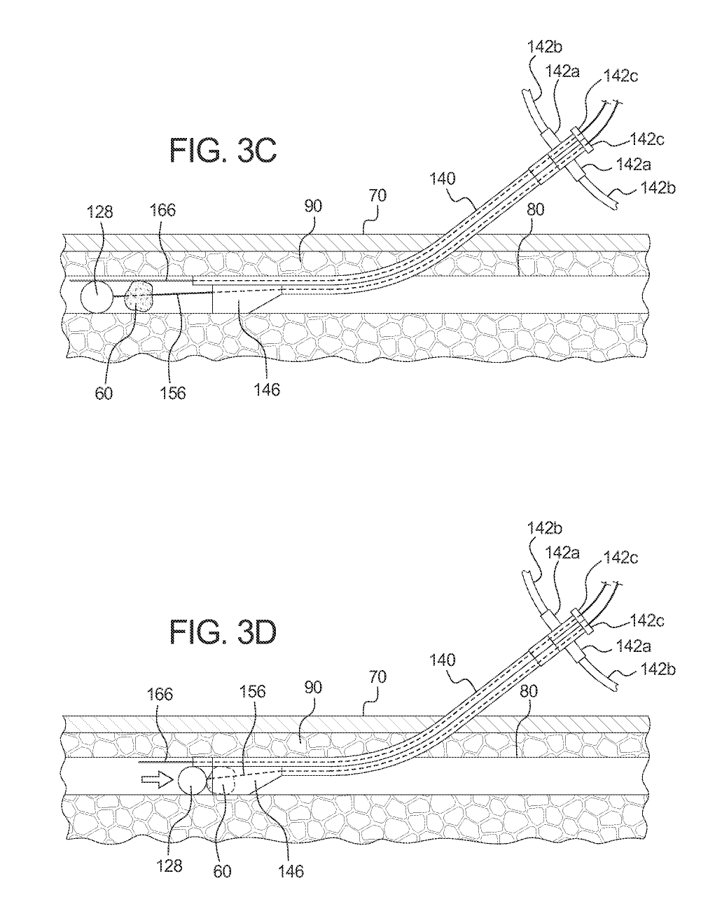

FIGS. 3A to 3D illustrate another embodiment of a medical device of the present disclosure. The embodiment of FIGS. 3A to 3D is similar to the embodiment illustrated at FIGS. each of the first and second inner passageways 140a, 140b includes, but differs primarily in that the sheath defines at least first and second inner passageways, and the flexible end portion of the sheath is located at an end section of one of the passageways. The inclusion of first and second inner passageways and a flexible end portion located at an end section of one of the passageways enables a wire to remain in the patient's blood vessel during the removal of the sheath (and the blood clot captured in the sheath), thereby allowing for better blood vessel control.

In particular, FIG. 3A illustrates a medical device of the present disclosure including a sheath 140 defining a first inner passageway 140a, and a second inner passageway 146b. Each of the first and second inner passageways 140a, 140b of sheath 140 includes a tube 142a and a port 142b coupled to a valve apparatus 142c. In different embodiments, sheath 140 includes a single valve apparatus for both passageways 140a, 140b. First inner passageway 140a includes a length that is longer than a length of the second inner passageway 140b. Flexible end portion 146 is located at an end section of second inner passageway 140b. In different embodiments, flexible end portion 146 is located at an end section of the first inner passageway 140a. It should be appreciated that the length of first and second inner passageways can be the same or different, and that the flexible end portion can be located at the shorter inner passageway or at the longer inner passageway. It should additionally be appreciated that in various embodiments, the first and second inner passageways have different inner diameters. For example, in an embodiment, longer inner passageway 140a has an inner diameter that is smaller than the inner diameter of longer inner passageway 140b.

FIG. 3B illustrates a point in time in which a wire 166 has been (i) inserted into first passageway 140a, (ii) one or more dilators have been inserted and removed from the sheath 140, and (iii) a wire 156 having an inflatable balloon actuator 128 coupled thereto has been inserted into second inner passageway 140b. Similar to the embodiment of FIGS. 1A to 1E, 2A to 2D, balloon actuator 128 can be actuated or inflated to cause the flexible end portion 146 of the second inner passageway 140b to flex and expand to a size to enable a blood clot to be captured and removed inside the expanded flexible portion 146. Balloon 128 is actuated by flowing air or gas through wire 156 similar to the manner in which balloon 52 and balloon 28 of dilator 20 are inflated in the embodiment of FIGS. 1A to 1E, 2A to 2D. Likewise, similar to the outward force on flexible end portion 46, the actuation or inflation of balloon 128 caused flexible end portion 146 of sheath 140 to expand outwardly (due to a thickness of flexible end portion), thereby exerting an outward force onto an inner surface of flexible end portion 146. The outward force flexed and expanded end portion 146 radially outwardly, thereby increasing the diameter of flexible end portion 146. At least a portion of the flexible end portion 146 accordingly expands to form a shape a generally cylindrical, conical or frusto-conical like shape as illustrated at FIG. 3C.

FIG. 3C illustrates a point in time when the flexible end portion has expanded from inflatable balloon actuator 128, and inflatable balloon actuator 128 has been moved to a position beyond the location of clot or debris 60. FIG. 3D illustrates wire 156 having been pulled backwardly (i.e., in the direction of arrow 96 at FIG. 3D), causing balloon 128 to pull and capture blood clot or debris 60 in the internal, expanded end portion 146 of sheath 140. With blood clot 60 captured at least substantially within the expanded flexible end portion 146 of sheath 140, sheath 140 and wire 156 can be pulled together out of the patient's skin to remove the blood clot 60 from the patient. Wire 166 remains in the patient's vessel while the sheath 140 (with the blood clot or debris 60 captured in the end portion) and wire 156 are being removed from blood vessel 80. The additional or second passageway of sheath 140 accordingly enables second wire 156 to remain in the blood vessel 80 so as to allow for better control of blood vessel 80. The additional second wire 156 remaining in the blood vessel allows a procedure to be easily repeated or for a slightly larger sheath to be placed into the blood vessel 80 to stop oozing or bleeding from the vessel. It should be appreciated that in an alternative embodiment, the sheath defines any suitable number of passageways greater than one, such as three, four or five passageways.

It should additionally be appreciated that the embodiment of FIGS. 3A to 3D including a sheath having more than one passageway does not need to be used for blood clot removal. For example, the multi-passageway sheath also provides a medical device that enables a user to insert one or more instruments into the first and/or the second inner passageways defined in the sheath. When the end portion of the multi-passageway sheath has not been expanded, the first and second passageways have a total combined diameter that is less than two separate sheaths having a combined total diameter approximately equal to the multi-passageway sheath in its expanded state.

It should accordingly be appreciated that an embodiment for a method of using the multi-passageway sheath of the present disclosure includes inserting a needle into a patient (using, for example, a 0.35 mm wire) and inserting a wire into the needle and into the blood vessel. The method further includes removing the needle from the patient, and placing both the multi-passageway sheath and a dilator over the wire and into the blood vessel using the wire as a guide. With the wire and dilator inserted into the sheath, the method further includes inserting an additional wire into the second passageway (e.g., inserted into the shorter passageway having the flexible end portion). An inflatable balloon that is coupled to the additional wire is actuated or inflated causing the flexible end portion to expand to a larger diameter due to the thickness of the flexible end portion. The dilator and both wires are removed from the sheath leaving the multi-passageway sheath inserted into the patient's blood vessel with two large passageways for a user (e.g., a cardiologist) to insert one or more instruments into the passageways so as to be able to perform various medical procedures on the patient. The multi-passageway sheath having a flexible end portion allows the sheath to be inserted into the patient in an unexpanded state, so as to allow a smaller insertion cite size relative to the insertion cite size of two separate sheaths having the same approximate combined diameter of the multi-passageway sheath in its expanded state.

It should further be appreciated that in alternative embodiments, the actuation of the flexible end portion 146 of sheath 140 can be performed using a balloon tipped dilator similar to the actuation structure and method in the embodiments illustrated at FIGS. 1A to 1E and FIGS. 2A to 2D. Likewise, the actuation or expansion of the flexible end portion 46 of the embodiment of FIGS. 1A to 1E and FIGS. 2A to 2D can be accomplished using a balloon tipped wire instead of a balloon tipped dilator. It should additionally be appreciated that the balloon actuator can be located at any portion of the wire and any portion of the dilator in any of the above described embodiments.

It should additionally be appreciated that the actuator of the present disclosure can be any suitable device or mechanism that allows the flexible end portion of the sheath to expand to a size that enables a balloon tipped wire to pull and capture the blood clot in the expanded flexible end portion, or to allow an instrument to be inserted into the expanded flexible end portion. It should also be appreciated that the flexible end portion of the sheath can be configured in any suitable manner that enables the end portion to expand to a shape and size that is sufficient to capture and remove most, or at least substantially all, of a blood clot from blood vessel. In one example, instead of the flexible end portion having a thickness that is suitable for expanding outwardly upon the exertion of the actuation or activation force, the flexible end portion has a plurality of pedals that splay or spread to form the expanded end portion upon an actuation force. In an embodiment, the pedals are joined together by a flexible web-like material that does not allow the blood clot or debris to pass through the material.

It should further be appreciated that dilator of the present disclosure can be any suitable size that enables the dilator to be inserted into, and moveable within, the sheath. Similarly, the sheath can be any suitable size that enables the sheath to be inserted into a patient's vein or artery.

It should additionally be appreciated that the sheath, the dilator and the wire of the present disclosure can be made of any medically suitable material. For example, in one embodiment, (i) the sheath and the dilator are at least substantially polymeric, and (ii) the wire is a flexible metal.

### Aspects of the Present Disclosure

Aspects of the subject matter described herein may be useful alone or in combination with one or more other aspect described herein. Without limiting the foregoing description, in a first aspect of the present disclosure, a medical device of the present disclosure includes a sheath having a size configured for insertion into a blood vessel. The sheath defines an inner passageway and includes a flexible end portion. The device further includes a dilator moveable within the inner passageway of the sheath, and an actuator configured to expand the flexible end portion of the sheath. The device further includes a wire moveable within the inner passageway of the sheath, and an inflatable balloon coupled to the wire. The inflatable balloon of the wire is configured such that when the balloon is inflated, the balloon can pull and capture a blood clot within the expanded flexible end portion of the sheath so that the blood clot can be removed from the blood vessel.

In accordance with a second aspect of the present disclosure, which can be used in combination with the first aspect, the actuator is an inflatable balloon coupled to the dilator, and wherein the sheath, the dialator and the balloon are configured such that inflation of the balloon causes the flexible end portion of the sheath to expand.

In accordance with a third aspect of the present disclosure, which can be used in combination with any one or more of the preceding aspects, the inflatable balloon is coupled to an end portion of the dilator.

In accordance with a fourth aspect of the present disclosure, which can be used in combination with any one or more of the preceding aspects, the flexible end portion has a length ranging from about 10 mm to 30 mm, and a thickness ranging from about 0.5 mm to 5 mm.

In accordance with a fifth aspect of the present disclosure, which can be used in combination with any one or more of the preceding aspects, the flexible end portion of the sheath includes a plurality of pedals configured to expand.

In accordance with a sixth aspect of the present disclosure, which can be used in combination with any one or more of the preceding aspects, the flexible end portion of the sheath has a thickness that is less than a thickness of a portion of the sheath adjacent to the flexible end portion.

In accordance with a seventh aspect of the present disclosure, which can be used in combination with any one or more of the preceding aspects, the dilator includes a first inner passageway configured to receive the wire, and a second inner passageway configured to receive pneumatic pressure to cause the actuator to expand the flexible end portion.

In accordance with an eighth aspect of the present disclosure, which can be used in combination with any one or more of the preceding aspects, the expanded flexible end portion forms at least in part a substantially cylindrical shape so as to contact an inner wall of the blood vessel.

In accordance with a ninth aspect of the present disclosure, which can be used in combination with any one or more of the preceding aspects, a sheath is sized and shaped for insertion into a blood vessel and defines an inner passageway extending along at least a portion of a length of the sheath, and the inner passageway is constructed and arranged to enable a medical instrument to be moveable with the inner passageway, the sheath including a flexible end portion having a thickness enabling the flexible end portion to expand.

In accordance with a tenth aspect of the present disclosure, which can be used in combination with any one or more of the preceding aspects, wherein the thickness of the flexible end portion enables the flexible end portion to expand to a size so as to allow a blood clot to be (i) captured within the expanded flexible end portion and (ii) removed from the blood vessel.

In accordance with an eleventh aspect of the present disclosure, which can be used in combination with the ninth or tenth aspect, wherein the flexible end portion has a length ranging from about 10 mm to 30 mm, and the thickness ranges from about 0.5 mm to 5 mm.

In accordance with a twelfth aspect of the present disclosure, which can be used in combination with the one or more of the preceding aspects aspect, the medical instrument is a dilator, and actuator is coupled to the dilator, and wherein the actuator is configured such that activating the actuator causes the flexible end portion of the sheath to expand.

In accordance with a thirteenth aspect of the present disclosure, which can be used in combination with any one or more of the preceding aspects, a balloon is coupled to the dilator, and the balloon configured to inflate so as to cause the flexible end portion of the sheath to expand.

In accordance with a fourteenth aspect of the present disclosure, which can be used in combination with any one or more of the preceding aspects, the medical device includes a wire and an inflatable balloon coupled to the wire.

In accordance with a fifteenth aspect of the present disclosure, which can be used in combination with any one or more of the preceding aspects, the wire is moveable within the inner passageway of the sheath and the inflatable balloon is configured such that when the balloon is inflated, a blood clot can be captured in the expanded flexible end portion of the sheath and removed from the blood vessel.

In accordance with a sixteenth aspect of the present disclosure, which can be used in combination with any one or more of the preceding aspects, the inner passageway defines a first passageway, and the sheath defines a second inner passageway, wherein the flexible end portion of the sheath is located at an end section of one of the first and second inner passageways.

In accordance with a seventeenth aspect of the present disclosure, which can be used in combination with any one or more of the preceding aspects, the inner passageway defines a first passageway, and the sheath defines a second inner passageway, and the second inner passageway includes a length that is less than the length of the sheath, and the flexible end portion is located at the end portion of the second inner passageway.

In accordance with an eighteenth aspect of the present disclosure, which can be used in combination with any one or more of the preceding aspects, a blood clot removing method includes inserting a sheath into a blood vessel, inserting a wire into the sheath, inflating a balloon coupled to the wire, expanding a flexible end portion of the sheath, pulling the wire along with the inflated balloon coupled to the wire so as to capture a blood clot in the expanded flexible end portion of the sheath, and pulling both the sheath and the wire together to remove the captured blood clot.

In accordance with a nineteenth aspect of the present disclosure, which can be used in combination with any one or more of the preceding aspects, the blood clot removing method includes inflating a balloon coupled to the dilator to expand the flexible end portion of the sheath.

In accordance with a twentieth aspect of the present disclosure, which can be used in combination with any one or more of the preceding aspects, the balloon for expanding the flexible end portion of the sheath is coupled to a dilator.

In accordance with a twenty-first aspect of the present disclosure, which can be used in combination with any one or more of the preceding aspects, the method includes which includes activating an actuator to expand the flexible end portion of the sheath.

In accordance with a twenty-second aspect of the present disclosure, which can be used in combination with any one or more of the preceding aspects, the method includes inserting a needle into the blood vessel to allow the sheath to be inserted into the blood vessel

In accordance with a twenty-third aspect of the present disclosure, which can be used in combination any one or more of the preceding aspects, the method includes expanding the flexible end portion of the sheath so that an outer surface of the expanded flexible end portion contacts an inner wall of the blood vessel.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A blood clot removing device comprising:
a sheath defining an inner passageway and including a flexible end portion, the sheath configured for insertion into a blood vessel;
a dilator moveable within the inner passageway of the sheath;
an actuator configured to expand the flexible end portion of the sheath;
a wire moveable within the inner passageway of the sheath; and
an inflatable balloon coupled to the wire, the inflatable balloon configured such that when the balloon is inflated, the balloon can pull and capture a blood clot within the expanded flexible end portion of the sheath so that the blood clot can be removed from the blood vessel.

2. The blood clot removing device of claim 1, wherein the actuator is an inflatable balloon coupled to the dilator, and wherein the sheath, the dilator and the inflatable balloon are configured such that inflation of the balloon causes the flexible end portion of the sheath to expand.

3. The blood clot removing device of claim 2, wherein the inflatable balloon actuator is coupled to an end portion of the dilator.

4. The blood clot removing device of claim 1, wherein the flexible end portion of the sheath has a length ranging from about 10 mm to 30 mm, and wherein the thickness ranges from about 0.5 mm to 5 mm.

5. The blood clot removing device of claim 1, wherein the flexible end portion of the sheath includes a plurality of pedals configured to expand.

6. The blood clot removing device of claim 1, wherein the flexible end portion of the sheath has a thickness that is less than a thickness of a portion of the sheath adjacent to the flexible end portion.

7. The blood clot removing device of claim 1, wherein the dilator includes a first inner passageway configured to receive the wire, and a second inner passageway configured to receive pneumatic pressure to cause the actuator to expand the flexible end portion.

8. The blood clot removing device of claim 1, wherein at least a portion of the expanded flexible end portion forms at least in part a substantially cylindrical shape that contacts an inner wall of the blood vessel.

9. A medical device comprising:
a sheath sized and shaped for insertion into a blood vessel, the sheath defining an inner passageway extending along at least a portion of a length of the sheath, the inner passageway constructed and arranged to enable a medical instrument to be moveable within the inner passageway, the sheath including a flexible end portion having a thickness enabling the flexible end portion to expand.

10. The medical device of claim 9, wherein the thickness of the flexible end portion enables the end portion to expand to a size to allow a blood clot to be (i) captured within the expanded flexible end portion and (ii) removed from the blood vessel.

11. The blood clot removing device of claim 10, wherein the flexible end portion includes a length ranging from about 10 mm to 30 mm, and wherein the thickness ranges from about 0.5 mm to 5 mm.

12. The blood clot removing device of claim 9, wherein the medical instrument is a dilator, and which includes an actuator coupled to the dilator, the actuator configured such that actuating the actuator causes the flexible end portion of the sheath to expand.

13. The blood clot removing device of claim 9, wherein the medical instrument is a dilator and which includes a balloon coupled to the dilator, the balloon configured to inflate so as to cause the flexible end portion of the sheath to expand.

14. The blood clot removing device of claim 9, which includes a wire and an inflatable balloon coupled to the wire.

15. The blood clot removing device of claim 14, wherein the wire is moveable within the inner passageway of the sheath and the inflatable balloon is configured such that when the balloon is inflated, a blood clot can be captured in the expanded flexible end portion of the sheath and removed from the blood vessel.

16. The blood clot removing device of claim 9, wherein the inner passageway is a first inner passageway and the sheath defines a second inner passageway, and wherein the flexible end portion of the sheath is located at an end section of one of the first and second inner passageways.

17. The blood clot removing device of claim 9, wherein the inner passageway is a first inner passageway and the sheath defines a second inner passageway, and wherein the first inner passageway extends along the length of the sheath, and the second inner passageway includes a length that is less than the length of the sheath, and wherein the flexible end portion is located at an end section of the second inner passageway.

18. A blood clot removing method comprising:
inserting a sheath into a blood vessel;
inserting a wire into the sheath:
inflating a balloon coupled to the wire;
expanding a flexible end portion of the sheath;
pulling the wire along with the inflated balloon coupled to the wire so as to capture a blood clot in the expanded flexible end portion of the sheath; and
pulling both the sheath and the wire together to remove the captured blood clot.

19. The blood clot removing method of claim 18, which includes inflating a balloon coupled to the dilator to expand the flexible end portion of the sheath.

20. The blood clot removing method of claim 18, wherein the balloon for expanding the flexible end portion of the sheath is coupled to a dilator.

21. The blood clot removing method of claim 18, which includes activating an actuator to expand the flexible end portion of the sheath.

22. The blood clot removing method of claim 18, which includes inserting a needle into the blood vessel to allow the sheath to be inserted into the blood vessel.

23. The blood clot removing method of claim 18, wherein expanding the flexible end portion of the sheath includes expanding the flexible end portion so that an outer surface of the expanded flexible end portion contacts an inner wall of the blood vessel.
